(19) United States
(12) Patent Application Publication
(10) Pub. No.: US 2006/0054169 A1
(43) Pub. Date: Mar. 16, 2006

(54) Respiration nozzle for a medical facemask

(76) Inventors: Tai-Kang Han, Tali City (TW); Hua Ting, Taichung (TW)

Correspondence Address: DELLETT & WALTERS, P. O. BOX 82788, PORTLAND, OR, 97282-0788, US

(21) Appl. No.: 10/942,588

(22) Filed: Sep. 15, 2004

(51) Int. Cl. A62B 7/10; A61M 15/08; A61M 16/00

(52) U.S. Cl. 128/207.18; 128/206.11

(57) ABSTRACT

A respiration nozzle has a hollow body with a front and a rear, at least one outlet made of a resilient membrane, and two inflatable nasal tubes. The at least one outlet is formed on the front of the hollow body and has a cross-shaped slit valve defined in the membrane to temporarily open and release air from the respiration nozzle. The two inflatable nasal tubes are formed on the rear of the hollow body and are enabled to inflate when air is pumped into the respiration nozzle. Therefore, air pressure inside the respiration nozzle is kept at a positive pressure to minimize consumption of electricity when pumping the air. Meanwhile, the inflatable nasal tubes gently and evenly match the nasal cavities without causing any uncomfortable feeling to the wearer.

### BACKGROUND OF THE INVENTION

1. Field of the Invention

The present invention relates to a respiration nozzle for a medical facemask, and more particularly to a respiration nozzle that perfectly matches nasal passages to make patients comfortable and constantly keeps a positive air pressure inside to efficiently save electricity when the respiration nozzle cooperates with a mobile respirator.

2. Description of Related Art

The human respiratory system includes lungs, an airway configured like the contour of a tree, and other thorax tissues. The airway tree contains a trachea extending from the pharynx and the larynx to gradually diverge into smaller and smaller airways such as bronchi, branch bronchi, small bronchi, and terminal bronchi in the lungs. Multiple alveolus are formed on the terminal bronchi to exchange gases in the lungs and of course under normal circumstances, people breathe effectively under varying conditions. However, several reasons cause problematic breathing. Take shortage of breath as an example, this happens when the airway is unusually constricted and then blocks the entry of air into the lungs. Serious shortage of breath can even cause life-threatening shock to a victim. Moreover, the respiratory system also provides protection from external factors such as bacteria, dust, toxic gases etc. Therefore, when the respiratory system does not work very well, patients need an air pre-treatment device to substitute the lungs to carry out the protection.

Conventional air pre-treatment devices (respirator) are only found in hospitals or other sanatoriums for smokers or patients having allergic respiratory conditions, hypersensitive rhinitis etc because impure air easily aggravates their lung conditions. Therefore, the air pre-treatment device mostly serves as an auxiliary device in formal medical treatment.

With reference to FIGS. 4 and 5, two conventional respiration nozzles in accordance with the prior art are used to cooperate with a respirator and are described in the following:

A first conventional respiration nozzle in FIG. 4 comprises a hollow body (50) with a front and a rear, two open outlets (52) formed on the front of the body (50), and two nasal tubes (54) formed on the rear of the body (50). Each the nasal tube (54) has a distal end and an annular lip (542) formed on the distal end to abut a periphery defining a patient's nasal cavity.

A second conventional respiration nozzle in FIG. 5 also comprises a hollow body (60) with a front and a rear, two open outlets (not shown) formed on the front of the body (60) and two dome-shaped nasal tubes (64) formed on the rear of the body (60). Each the nasal tube (64) has a short tube (642) adapted to extend into a patient's nasal passage.

However, the first and second respiration nozzles have the following drawbacks:
1. In normally operation situation, the air pre-treatment device is kept in a positive air pressure in the respiration nozzle that is higher than atmosphere out of the respiration nozzle. The open outlets (52) make air pressure inside the conventional respiration nozzles balance with the atmosphere and decrease. For a patient suffering respiratory illness, terminal bronchi usually accumulate phlegm inside or injured to have scabs. When the patient breathes out, the terminal bronchi usually collapse. Therefore, the respirator has to keep a positive pressure all the time to distend the terminal bronchi to maintain respiration. Therefore, the respirator consumes a significant amount of electricity in order to constantly pump air into the conventional respiration nozzles to maintain the positive pressure.
2. The nasal tubes (54, 64) of the conventional respiration nozzles have to extend into the nasal cavities and abut tightly the inner surface of the nasal cavities to avoid air leakage. Therefore, the patient feels uncomfortable because the inner surfaces of the nasal cavities become stimulated and generate secretions or are injured by rubbing with the nasal tubes (54, 64), such that crusts form on the inner surface of the nasal cavities. The nasal tubes (54, 64) that protrude and abut the inner surfaces of the nasal cavities can not perfectly match the nasal cavities so that turbulence is usually generated, thereby causing insufficient breath to the patient and more noise is generated while the patient is breathing.

The present invention has arisen to mitigate or obviate the disadvantages of the conventional respiration nozzles for a medical facemask.

### SUMMARY OF THE INVENTION

One objective of the present invention is to provide a respiration nozzle that is comfortable and makes no noise in use.

The other objective of the present invention is to provide a respiration nozzle that keeps a constant positive pressure in the respiration nozzle to make a respirator economic in its consumption of electricity.

To achieve the above-mentioned objective, the respiration nozzle in accordance with the present invention comprises a hollow body with a front and a rear, at least one outlet made of a membrane, and two inflatable nasal tubes. The at least one outlet is formed on the front of the hollow body and has a cross-shaped slit valve defined in a center of the membrane to temporarily open and release air from the respiration nozzle. The two inflatable nasal tubes are formed on the rear of the hollow body and are able to inflate when air is pumped into the respiration nozzle. Therefore, air pressure inside the respiration nozzle is kept at positive pressure to save electricity when pumping the air. Meanwhile, the inflatable nasal tubes gently and evenly match with the nasal cavities without causing any uncomfortable feeling.

Further benefits and advantages of the present invention will become apparent after a careful reading of the detailed description with appropriate reference to the accompanying drawings.

### BRIEF DESCRIPTION OF THE DRAWINGS

FIG. 1 is a perspective view of a respiration nozzle for a medical facemask in accordance with the present invention;

FIG. 2 is an operational side view of the respiration nozzle in accordance with FIG. 1;

FIG. 3 is an operational perspective view of the respiration nozzle of FIG. 1, wherein the respiration nozzle is in combination with a facemask;

FIG. 4 is a perspective view of a first conventional respiration nozzle in accordance with the prior art; and

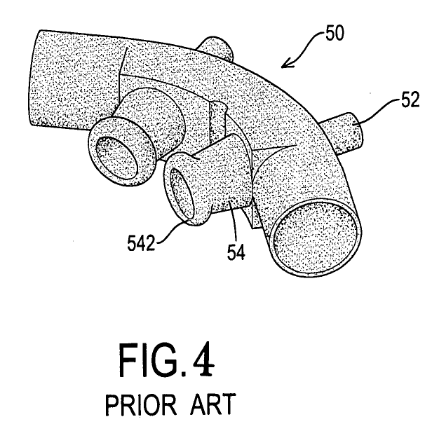

FIG. 5 is a perspective view of a second conventional respiration nozzle in accordance with the prior art.

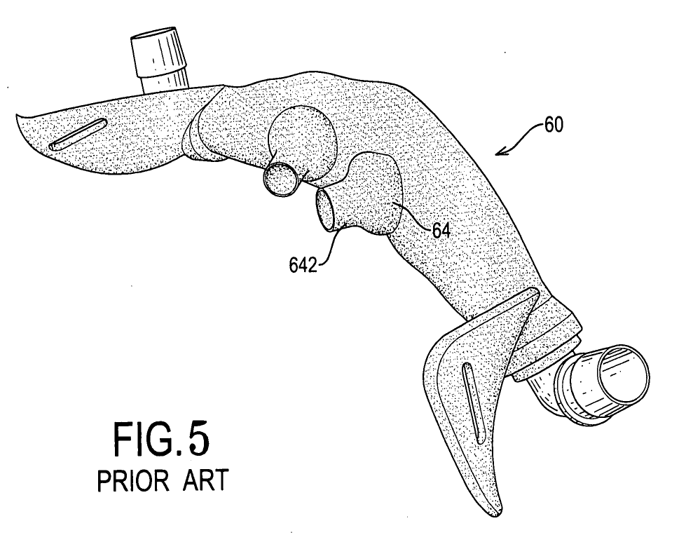

### DETAILED DESCRIPTION OF THE PREFERRED EMBODIMENT

A respiration nozzle for a medical mask in accordance with the present invention comprises a hollow body with a front and a rear, at least one outlet made of a membrane, and two inflatable nasal tubes. The at least one outlet is formed on the front of the hollow body and has a cross-shaped slit valve defined in a center of the membrane to temporarily open and then release air from the respiration nozzle. The two inflatable nasal tubes are formed on the rear of the hollow body and are able to inflate when air is pumped into the respiration nozzle. Therefore, air pressure inside the respiration nozzle is kept in a positive pressure mode to minimize electricity consumption when pumping the air. Meanwhile, the inflatable nasal tubes gently and evenly match the nasal cavities without causing any uncomfortable feeling.

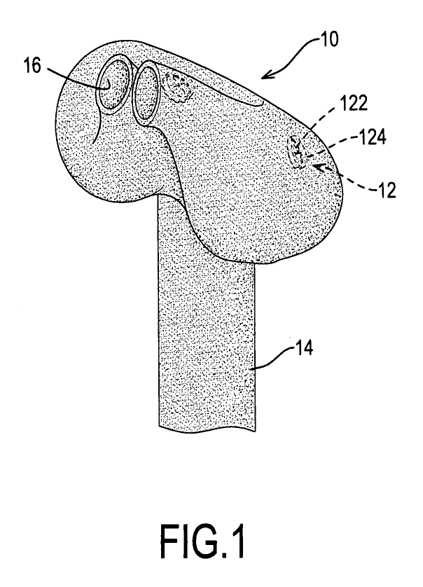

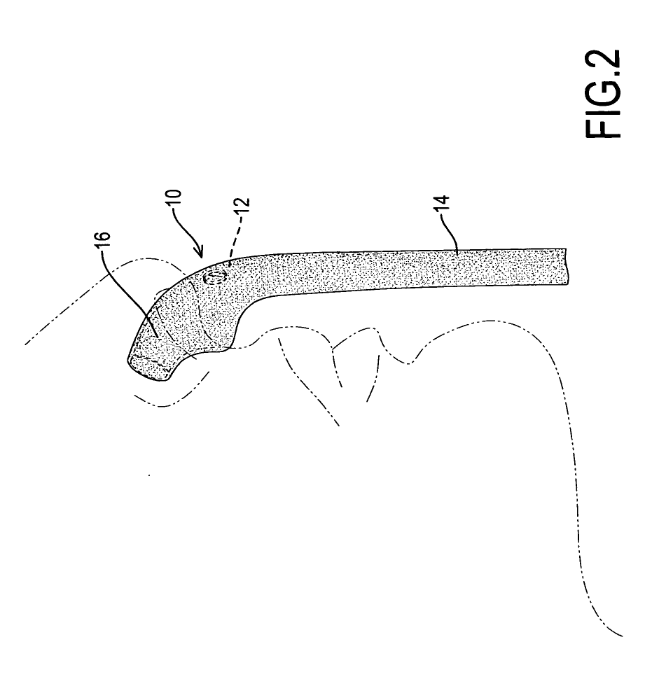

With reference to FIGS. 1 and 2, a preferred embodiment of the respiration nozzle in accordance with the present invention has a hollow body (10), two outlets (12), an inlet tube (14), and two inflatable nasal tubes (16).

The hollow body (10) is oval and has a front, a rear and a bottom. The two outlets (12) are formed on the front of the hollow body (10) and each outlet (12) is made of a round resilient membrane with an slit valve. The round membrane has a center, an inner face and a circular flange (122). Preferably, the slit valve is a cross-shaped slit valve defined in the center of the membrane. The circular flange (122) is formed on the inner face around the round membrane. The membrane (122) is closed in an ordinary situation when the respirator operates in a positive pressure. However, the slit valve opens the membrane to release air when the patient breathes to increase the air pressure inside the respiration nozzle. After releasing the over-pressurized air, the slit valve of the membrane closes again to keep the air pressure effectively positive. Therefore, the respiratory has no need to continuously pump air into the respiration nozzle to keep the air pressure and thus to efficiently saves electricity.

The inlet tube (14) is formed on the bottom of the hollow body (10) and adapted to connect with the respirator. Preferably, the inlet tube (14) is connected to a respirator with air heating, filtering and moisturizing devices.

The two inflatable nasal tubes (16) are formed on the front of the hollow body (10) and made of soft material in a shape corresponding to patient's nasal cavities. Preferably, each inflatable nasal tube (16) has a narrowing distal end. With particular reference to FIG. 2, when the respirator pumps air into the respiration nozzle, the inflatable nasal tubes (16) are inflated to evenly and correspondingly match with the nasal cavities. Because the inflatable nasal tubes are made of soft material, the respiration nozzle does not cause any uncomfortable feeling to the patient. Therefore, the patient's nasal cavities do not generate abnormal secretion and the patient can breathe sufficiently. Meanwhile, the hollow body (10) and the inflatable nasal tubes (16) are formed together in a streamline single-piece molding so that no turbulence is generated thus avoiding noise and/or insufficient breath to the patient.

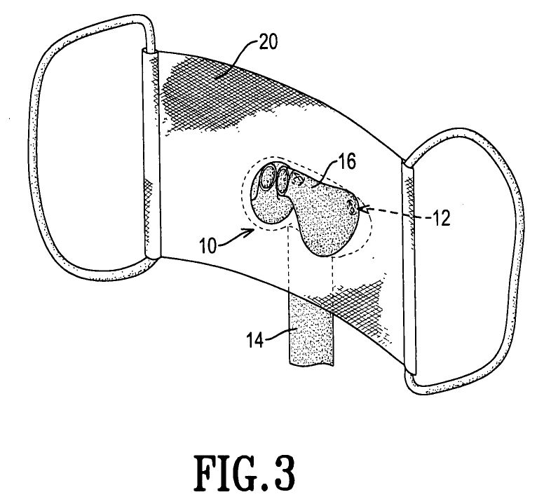

With reference to FIG. 3, another embodiment of the respiration nozzle in accordance with the present invention cooperates with a facemask (20). The facemask (20) has a front and a rear and the hollow body (10) is attached inside the facemask (20). The two outlets (12) are exposed on the front facemask (20) and the two inflatable nasal tubes (16) are extended out of the rear of the facemask (20) to engage with the patient's nasal cavities. The facemask (20) provides a positioning efficiency to the respiration nozzle and further provides a more pleasant appearance of the respiration nozzle because the patients mostly do not want to be regarded as having serious illness.

According to the above description, the respiration nozzle has the following advantages:
1. The respiration nozzle can keep an effective positive pressure because the outlets (12) made of resilient membrane only temporarily release excessive air pressure to make the effective positive pressure constant. Therefore, the respirator is enabled to function at an economic level of electricity consumption.
2. Patients feel comfortable because the inflatable nasal tubes (16) softly and smoothly match the nasal cavities and cause neither noise nor disturbance of breath.

Although the invention has been explained in relation to its preferred embodiments, many other possible modifications and variations can be made without departing from the spirit and scope of the invention as hereinafter claimed.

What is claimed is:

1. A respiration nozzle for a medical facemask comprising:
a hollow body (10) with a front, a rear, a side and a bottom;
an inlet tube (14) in communication with the hollow body (10), wherein the inlet tube (14) is formed on the bottom of the hollow tube (10);
two outlets (12) formed on the front of the hollow body (10), wherein each outlet (12) is made of round resilient membrane (122) and has a center, an inner face, a cross-shaped slit valve defined in the center, and an annular flange (124) formed on the inner face around the round resilient membrane (122); and
two inflatable nasal tubes (16) formed on the rear of the hollow body (10) and made of soft material and shaped to adapt to nasal cavities.

2. The respiration nozzle as claimed in claim 1, wherein the hollow tube (10) cooperates with a facemask (20) with a front and a rear;
wherein the two outlets (12) are exposed on the front of the facemask (20) and the two inflatable nasal tubes (16) extend from the rear of the facemask (20).

3. The respiration nozzle as claimed in claim 1, wherein the hollow tube (10) cooperates with a facemask (20) with a front and a rear;
wherein the two outlets (12) are exposed on the front of the facemask (20) and the two inflatable nasal tubes (16) extend from the rear of the facemask (20).

4. The respiration nozzle as claimed in claim 3, wherein each inflatable nasal tube (16) has a narrowing distal end.
